(19) United States
(12) Patent Application Publication
(10) Pub. No.: US 2021/0388065 A1
(43) Pub. Date: Dec. 16, 2021

(54) ANTIBODIES TO SARS-CORONAVIRUS (COVID-19) S1 SPIKE PROTEIN

(71) Applicants: ACTIVE MOTIF SHANGHAI LIMITED, Shanghai (CN); FUDAN UNIVERSITY, Shanghai (CN); SHANGHAI PUBLIC HEALTH CLINICAL CENTER, Shanghai (CN)

(72) Inventors: Yanan LU, Shanghai (CN); Fei LAN, Shanghai (CN); Jianqing XU, Shanghai (CN); Xi ZHAO, Shanghai (CN); Longfei DING, Shanghai (CN); Yongheng WANG, Shanghai (CN); Jinkai WAN, Shanghai (CN); Shenghui XING, Shanghai (CN); Joseph FERNANDEZ, Carlsbad, CA (US)

(73) Assignees: ACTIVE MOTIF SHANGHAI LIMITED, Shanghai (CN); SHANGHAI PUBLIC HEALTH CLINICAL CENTER, Shanghai (CN); FUDAN UNIVERSITY, Shanghai (CN); SHANGHAI PUBLIC HEALTH CLINICAL CENTER, Shanghai (CN)

(21) Appl. No.: 17/053,008

(22) PCT Filed: Apr. 15, 2020
(86) PCT No.: PCT/CN2020/084857

§ 371 Date: Jul. 20, 2021

(51) Int. Cl. C07K 16/10; G01N 33/569

(52) U.S. Cl. CPC C07K 16/10; C07K 2317/34; C07K 2317/565; G01N 33/56983

(57) ABSTRACT

The present invention provides recombinant monoclonal antibodies that bind to the Severe Acute Respiratory Syndrome-Coronavirus-2 (SARS-CoV-2 or COVID-19) spike protein, and methods of use. In various embodiments of the invention, the antibodies are fully human antibodies that bind to SARS-CoV-2 spike protein. In some embodiments, the antibodies of the invention are useful for inhibiting or neutralizing SARS-CoV-2 activity, thus providing a means of treating or preventing COVID-19 infection in humans. In some embodiments, the invention provides for a combination of one or more antibodies that bind to the SARS-CoV-2 spike protein for use in treating COVID-19 infection. In certain embodiments, the one or more antibodies bind to distinct non-competing epitopes comprised in the receptor binding domain of the SARS-CoV-2 spike protein. In certain embodiments, the antibodies of the invention can be used to make an in vitro diagnostic for detection of COVID-19 in human and non-human animal samples In still other embodiments the antibodies of the present invention can be used to remove virus from patient plasma via plasmapheresis.

### FIELD OF THE INVENTION

The present invention is related to human antibodies and antigen-binding fragments of human antibodies that specifically bind to the spike protein of the Severe Acute Respiratory Syndrome-Coronavirus-2 (SARS-CoV-2), and therapeutic and diagnostic methods of using those antibodies.

### STATEMENT OF RELATED ART

Severe Acute Respiratory Syndrome-Coronavirus-2 (SARS-CoV-2, COVID-19) is a newly emergent betacoronavirus which causes severe acute respiratory disease. As of Mar. 28, 2020, the World Health Organization reported 509,164 cases of COVID-19 worldwide and 23,335 deaths, yielding a global death rate from the disease of 4.58%. Over 100,000 COVID-19 cases have been identified in the United States with 1,603 confirmed deaths, yielding a national COVID-19 death rate of 1.57%. At this rate, if the virus continues unabated despite clinical and public health interventions, COVID-19 could claim as many as 5 million lives in the United States alone. Clinical features of SARS-CoV-2 infection in humans range from an asymptomatic infection to very severe pneumonia, with potential development of acute respiratory distress syndrome, septic shock and multi-organ failure resulting in death.

SARS-CoV-2 shares substantial genetic and functional similarity with other pathogenic human betacoronaviruses, including Severe Acute Respiratory Syndrome Coronavirus (SARS-CoV) and Middle Eastern Respiratory Syndrome Coronavirus (MERS-CoV). The virus is believed to have originated in bats (Ge et al., 2013) or pangolins (Zhang et al., 2020); the exact source and animal reservoir of COVID-19 is yet to be elucidated.

COVID-19 utilizes an extensively glycosylated envelope-bound homotrimeric Spike protein (S) to interact with the cellular ACE2 receptor. Binding to ACE2 triggers a series of cell membrane fusion events resulting in viral entry (Luan et al., 2020; Ortega et al., 2020). Each S protomer consists of two subunits: a globular 51 domain at the N-terminal region, and the membrane-proximal S2 and transmembrane domains. Determinants of host range and cellular tropism are found in the ACE2 receptor binding domain (RBD) within the 51 domain, while mediators of membrane fusion have been identified within the S2 domain. A recent high-resolution structure of SARS-CoV-2 RBD bound to the N-terminal peptidase domain of ACE2 suggests that the overall ACE2-binding mechanism is virtually the same between SARS-CoV-2 and SARS-CoV RBDs, indicating convergent ACE2-binding evolution between these two viruses. This suggests that disruption of the RBD and ACE2 interaction would block SARS-CoV-2 entry into the target cell. Targeting of ACE2 has been shown, in fact, to inhibit COVID-19 infection. However, given the important physiological roles of ACE2 in vivo, these agents may have undesired side effects. Anti-Spike antibodies, on the other hand, should permit inhibition of COVID-19-ACE2 interactions without alternating or impeding ACE2 function. Furthermore, SARS-CoV or MERS-CoV RBD-based vaccine studies in experimental animals have also shown strong polyclonal antibody responses that inhibit viral entry.

These important findings indicate that anti-Spike antibodies should be able to effectively block SARS-CoV-2 entry.

U.S. Pat. No. 9,718,872 B2 describes the isolation and characterization of human antibodies to Middle East Respiratory Syndrome coronavirus spike protein as do U.S. Pat. No. 10,131,704B2, U.S. Pat. No. 10,406,222B2, and WO2016138160A1 while EP-2193802-B1, WO2008060331A9, U.S. Pat. No. 7,629,443B2, WO2006095180A2 describe the generation of neutralizing antibodies to SARS-CoV. Neutralizing human antibodies to the receptor binding domain of the COVID-19 spike protein from eight SARS-COV-2 infected individuals have been disclosed in a bioRxiv preprint by Ju et al., doi: https://doi.org/10.1101/2020.03.21.990770.

Thus far, there has been no vaccine or therapeutic agent to prevent or treat COVID-19 infection. In view of the continuing and substantial threat to human health and high fatality rate (over 4.5% globally), there is an urgent need for preventive and therapeutic antiviral therapies for COVID-19 control. Fully human antibodies that specifically bind to SARS-CoV-2 spike protein with high affinity and inhibit virus infectivity could be important in the prevention and treatment of COVID-19 infection. Furthermore, high specificity COVID-19 Spike antibodies could serve as important reagents for the development of rapid in vitro diagnostics for COVID-19 detection at all stages of infection and, when applicable, therapeutic intervention.

### BRIEF SUMMARY OF THE INVENTION

Provided herein are antibodies and fragments thereof that bind to SARS-CoV-2 spike protein, as well as nucleic acids encoding these antibodies, methods of use and articles containing them. Exemplary sequences of heavy and light chain regions that comprise CDRs useful in binding SARS-CoV-2 spike protein are provided in the Sequence Listing and the Figures.

### BRIEF DESCRIPTION OF THE DRAWINGS

FIG. 1. COVID-19 antibody Heavy Chain Variable Regions (HCVR). Alignment of nine patient-derived COVID-19 antibody HCVR amino acid sequences. Asterisks represent fully conserved residues, colons indicate conservation between groups of strong similar chemical properties, and periods indicate conservation between groups of weakly similar properties.

FIG. 2. COVID-19 antibody Light Chain Variable Regions (LCVR). Alignment of nine patient-derived COVID-19 antibody LCVR amino acid sequences. Asterisks represent fully conserved residues, colons indicate conservation between groups of strong similar chemical properties, and periods indicate conservation between groups of weakly similar properties.

FIG. 3. SARS-CoV-2 Spike protein (Receptor Binding Domain, RBD) binding assay with recombinant human COVID-19 antibodies. A microtiter plate-based assay used to detect the interaction between SARS-CoV-2 Spike protein RBD with COVID-19 antibodies of the present invention. SARS-CoV-2 Spike RBD was coated onto microtiter plates and then incubated with graded doses of recombinant human antibodies. Bound antibodies were detected with anti-human antibodies conjugated to horse radish peroxidase. Detection of COVID-19 antibodies was achieved upon addition of HRP substrate and measuring the resulting absorbance at 450 nm. EC50 values for the representative antibodies of the present invention are given under each antibody identifier.

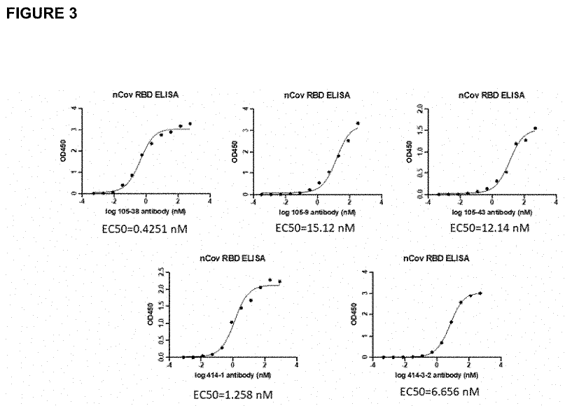

FIG. 4. Inhibition of COVID-19 Spike RBD/angiotensin converting enzyme-2 (ACE-2) interactions by recombinant human anti-COVID-19 antibodies. Using the plate-based assay described in FIG. 3, binding of a constant quantity of recombinant ACE-2 in the presence of a graded dose of anti-COVID-19 antibodies was performed to determine the IC50 for each antibody of the present invention.

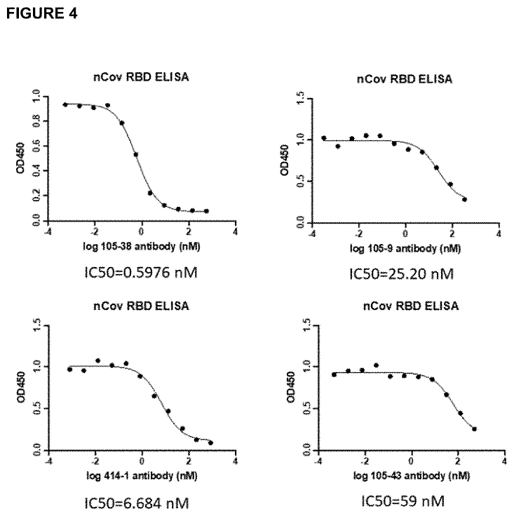

FIG. 5. Anti-COVID-19 antibodies of the present invention are neutralizing antibodies, inhibiting cellular infection. Cell-based infection assays using A549 lung epithelial cell line and Vero kidney epithelial cell line expressing the ACE2 receptor and pseudotyped SARS-CoV-2 virus bearing the S1 spike protein show strong inhibition of viral infection by antibodies of the present invention.

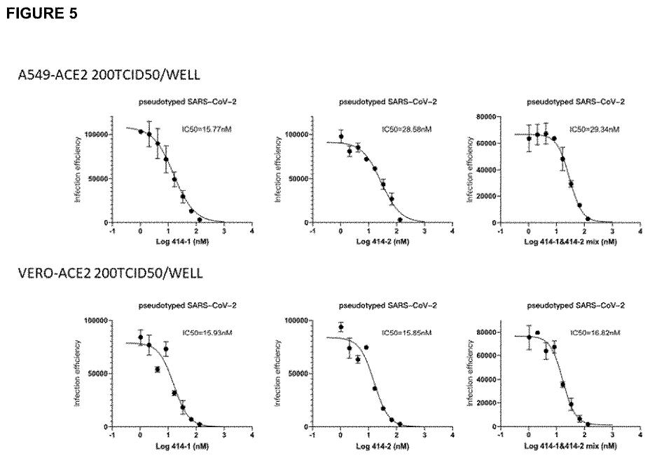

FIG. 6. An exemplary filter device comprising elongated tube 601 and internally disposed microfilters 605, with entry 607 and exit 609.

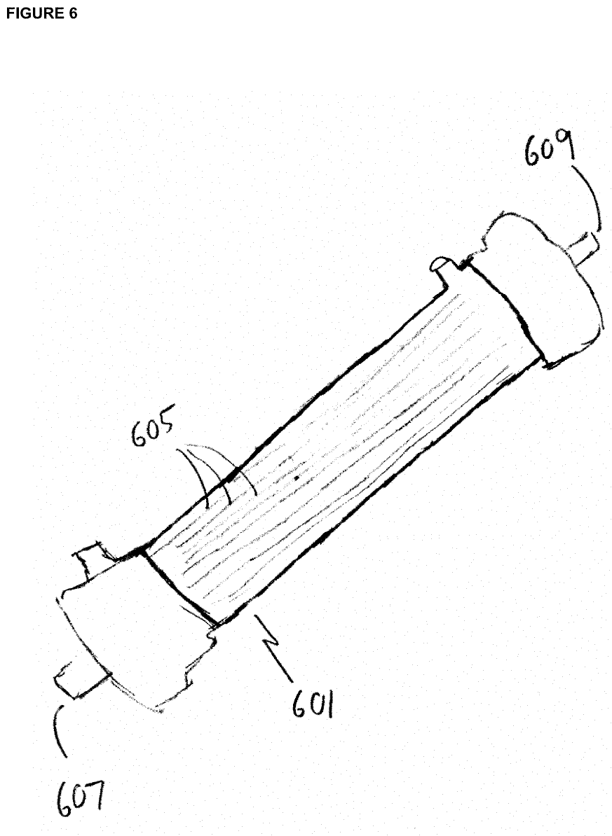

### DETAILED DESCRIPTION OF THE INVENTION

### I. Definitions

As used herein, the term “immunoglobulin” refers to a polypeptide encoded by a member of the immunoglobulin gene superfamily. This includes both immunoglobulin heavy chains and immunoglobulin light chains.

As used herein, the term “antibody” refers to an immunoglobulin molecule that recognizes and specifically binds to a one or more target antigens. The term “antibody” includes both intact antibodies and antigen-binding fragments of antibodies. An “intact antibody” comprises a tetramer composed of two pairs of polypeptide chains, each pair having one “light” chain (about 25 kD) and one “heavy” chain (about 50-70 kD) held together through disulfide bonds. Light chains and heavy chains each comprise a variable region and a constant region.

Antibody binding occurs through at least one antigen recognition site within the variable region of the immunoglobulin at one or more epitopes on the antigen. The antigen recognition site of the variable region is composed of hypervariable regions or complementarity determining regions (“CDRs”) and frameworks regions. Each light chain and heavy chain of an intact immunoglobulin typically comprises three CDRs referred to as HCDRI, HCDR2 and HCDR3 (heavy chain) and (HCDRI, HCDR2 and HCDR3 (light chain).

Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The lights chains can be either lambda or kappa.

As used herein, the term “intact” in reference to an antibody refers to an antibody comprising two heavy chains and two light chains.

As used herein, the term “antigen-binding fragment” in reference to an antibody is any fragment of an antibody that binds a target antigen. Antigen-binding fragments of antibodies typically include at least a heavy chain variable region and a light chain variable region. Antigen-binding fragments include, without limitation, the following forms: Fv (a monovalent or bi-valent variable region fragment, and can encompass only the variable regions (e.g., VL and/or VH), Fab (VLCL VHCH), F(ab′)2, Fv (VLVH), scFv (single chain Fv) (a polypeptide comprising a VL and VH joined by a linker, e.g., a peptide linker), (scFv)2, sc(Fv)2, bispecific sc(Fv)2, bispecific (scFv)2, minibody (sc(FV)2 fused to CH3 domain), triabody is trivalent sc(Fv)3 or trispecific sc(Fv)3.

The term antibody further embraces multivalent antibodies (antibodies comprising binding regions that bind two different epitopes or proteins).

As used herein, the term “monoclonal antibody” refers to a clonal preparation or composition of antibodies with a single binding specificity and affinity for a given epitope on an antigen (“monoclonal antibody composition”). A “polyclonal antibody” refers to a preparation or composition of antibodies that are raised against a single antigen, but with different binding specificities and affinities (“polyclonal antibody composition”).

As used herein, the term “chimeric antibody” refers to an antibody having amino acid sequences derived from two or more species. In one embodiment, the variable region of both light and heavy chains correspond to the variable region of antibodies derived from one species of mammal (e.g., mouse, rat, rabbit, etc.) with the desired specificity, affinity and capability, while the constant region are homologous the sequence derived from another species (typically in the subject receiving the therapy, e.g., human) to avoid eliciting an immune response.

As used herein, the term “humanized antibody” refers to a chimeric antibody in which the CDRs, obtained from the VH and VL regions of a non-human antibody having the desired specificity, affinity and capability are grafted to a human framework sequence. In one embodiment, the framework residues of the humanized antibody is modified to refine and optimize the antibody specificity, affinity and capability. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996).

As used herein, the term “human antibody” refers to an antibody produced by a human or an antibody having an amino acid sequence corresponding thereto made by any technique known in the art.

As used herein, the term “hybrid antibody” refers to antibody in which pairs of heavy and light chains form antibodies with different antigenic determinant regions are assembled together so that two different epitopes or two different antigens can be recognized and bound by the resulting tetramer. Hybrid antibodies can be bispecific (binding 2 distinct antigens or epitopes) or multispecific (>1 distinct antigen or epitope).

As used herein, an antibody is “monospecific” if all of its antigen binding sites bind to the same epitope.

As used herein, an antibody is “bispecific” if it has at least two different antigen binding sites which each bind to a different epitope or antigen.

As used herein, an antibody is “polyvalent” if it has more than one antigen binding site. For example, an antibody that is tetravalent has for antigen binding sites.

Three complementarity determining regions contained within a variable region sequence refers to the set of CDR's including CDR1, CDR2 and CDR3 contained within the variable region sequence. It can refer to the CDR sequence set included in a heavy chain (HCDRI, HCDR2 and HCDR3) or a light chain (LCDRI, LCDR2 and LCDR3). A “full” CDR sequence set refers to the state including both heavy chain and light chain CDR sequences.

The specificity of the binding can be defined in terms of the comparative dissociation constants (Kd) of the antibody (or other targeting moiety) for target, as compared to the dissociation constant with respect to the antibody and other materials in the environment or unrelated molecules in general. A larger (higher) Kd is a Kd that describes a lower affinity interaction. Conversely a smaller (lower) Kd is a Kd that describes a higher affinity interaction or tighter binding. By way of example only, the Kd for an antibody specifically binding to a target may be femtomolar, picomolar, nanomolar, or micromolar and the Kd for the antibody binding to unrelated material may be millimolar or higher. Binding affinity can be in the micromolar range (kD=10⁻⁴ to 10⁻⁶), nanomole range (kD=10⁻⁷ M to 10⁻⁹ M), picomole range (kD=10⁻¹⁰ M to 10⁻¹² M), or femtomole range (kD=10⁻¹³ M to 10⁻¹⁵ M).

As used herein, an antibody “specifically binds” or is “specific for” a target antigen or target group of antigens if it binds the target antigen or each member of the target group of antigens with an affinity of at least any of 1×10⁻⁶ M, 1×10⁻⁷M, 1×10⁻⁸M, 1×10⁻⁹ M, 1×10⁻¹⁰ M, 1×10⁻¹¹ M, 1×10⁻¹² M, and binds to the target antigen with an affinity that is at least two-fold greater than its affinity for non-target antigens. Typically, specific binding is characterized by binding the antigen with sufficient affinity that the antibody is useful as a diagnostic to detect the antigen or epitope and/or as a therapeutic agent in targeting the antigen or epitope.

As used herein, an antibody “binds” or “recognizes” an antigen or epitope if it binds the antigen or epitope with a Kd of less than 10⁻⁴M (i.e., in the micromolar range).

As used herein, and antibody “neutralizes” a virus if it both binds to the virus and inhibits infectivity of the virus.

As used herein, and antibody “interacts with” amino acid residues if the amino acid residues are included in an epitope to which the antibody binds.

As used herein, and antibody “blocks” or “antagonizes” the binding between two molecules when it competitively reduces or prevents interaction of the molecules. In an embodiment, the measured level of reduction can be at least any of 5%, 10%, 25%, 50%, 80%, 90%, 95%, 97.5%, 99%, 99.5%, 99.9% of a control.

As used herein, the terms “antigen,” “immunogen,” and “antibody target,” refer to a molecule, compound, or complex that is recognized by an antibody, i.e., can be bound by the antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “pharmaceutical composition” refers to a composition comprising a pharmaceutical compound (e.g., a drug) and a pharmaceutically acceptable carrier.

As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

As used herein, the term “diluent” refers to a pharmaceutically acceptable carrier which does not inhibit a physiological activity or property of an active compound, such as an antibody, or immunoconjugate, to be administered and does not irritate the subject and does not abrogate the biological activity and properties of the administered compound. Diluents include any and all solvents, dispersion media, coatings, surfactants, antioxidants, preservative salts, preservatives, binders, excipients, disintegration agents, lubricants, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Pluronics, Triton-X, or polyethylene glycol (PEG).

As used herein, the term “subject” refers to an individual animal, including, without limitation, animals that may be reservoirs of SARS-CoV2. The term “patient” as used herein refers to a subject under the care or supervision of a health care provider such as a doctor or nurse. Subjects include mammals, including, without limitation, humans, pangolins, bats, civets, camels.

As used herein, the term “biological sample” refers to a sample of fluid or tissue sample derived from a subject. Biological samples include, without limitation, body fluids, tissues, secretions, and waste products derived from a subject, such blood, plasma, serum, tears, saliva, cerebrospinal fluid, amniotic fluid, exhaled breath condensate, urine and feces.

As used herein, the term “immunoassay” refers to a method of detecting an analyte by detecting binding between an antibody binding fragment of an antibody and the analyte. Immunoassay methods include, for example, radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS).

An exemplary amino acid sequence of COVID19 Spike Glycoprotein Receptor Binding Domain is provided at SEQ ID NO. 41.

As used herein, the term “kit” refers to a collection of items intended for use together. The items in the kit may or may not be in operative connection with each other. A kit can comprise, e.g., antibodies or antigen-binding fragments as disclosed herein, optionally attached to a solid support, as well as reagents for performing assays and control reagents. Typically, items in a kit are contained in primary containers, such as vials, tubes, bottles, boxes or bags. Separate items can be contained in their own, separate containers or in the same container. Items in a kit, or primary containers of a kit, can be assembled into a secondary container, for example a box or a bag, optionally adapted for commercial sale, e.g., for shelving, or for transport by a common carrier, such as mail or delivery service.

As used herein, the term “solid support” refers to a solid material to which antibodies can be attached. Exemplary solid supports include, without limitation, beads or particles (e.g., made of, sepharose), microtiter plates, microchips, filters, membranes or fibers, e.g., microfibers (e.g., made of polyethylene, vinyl alcohol copolymer or polysulfone) and. Hollow microfibers for use in plasmapheresis can have an inner diameter of about 100 microns and 400 microns and a wall thickness of about 30 microns to 60 microns.

In one embodiment, provided herein is a filter device for filtering blood during plasmapheresis. The device comprises an elongated, hollow tube comprising and entrance and an exit and, within the tube, a plurality of elongated microporous fibers, typically having an interior lumen extending along the length thereof, wherein the microporous filters comprise, immobilized thereto, an antibody or antigen binding fragment that binds to a SARS-CoV-2 spike protein, as described herein.

Antibodies and antigen-binding fragments as disclosed herein are useful to detect SARS-CoV-2 infection and to make a diagnosis of SARS-CoV-2 infection or COVID 19 disease. As used herein, the term “diagnosis” refers to a relative probability that a subject has a disorder. Similarly, the term “prognosis” refers to a relative probability that a certain future outcome may occur in the subject.

As used herein, the term terms “therapy,” “treatment,” “therapeutic intervention” and “amelioration” refer to any activity resulting in a reduction in the severity of symptoms or disease. The terms “treat” and “prevent” are not intended to be absolute terms. Treatment and prevention can refer to any delay in onset, amelioration of symptoms, improvement in patient survival, increase in survival time or rate, etc. Treatment and prevention can be complete or partial. In some aspects, the severity of disease is reduced by at least 25%, 50%, 75%, 80%, or 90%, or in some cases, no longer detectable using standard diagnostic techniques.

As used herein, the terms “effective amount,” “effective dose,” and “therapeutically effective amount,” refer to an amount of an agent, such as an antibody or antigen-binding fragment, that is sufficient to generate a desired response, such as reduce or eliminate a sign or symptom of SARS-CoV-2 infection or COVID 19 disease. In some examples, an “effective amount” is one that treats (including prophylaxis) one or more symptoms and/or underlying causes of any of a disorder or disease and/or prevents progression of a disease.

The term “recombinant DNA” or “recombinant nucleic acid” refers to a nucleic acid molecule comprising sequences not normally attached in nature. The term “recombinant cell” refers to a cell comprising a recombinant nucleic acid or a nucleic acid not normally found in the cell in nature. The term “recombinant” when used in reference to a protein, refers to a protein produced by a recombinant DNA technology.

As used herein, the term “expression construct” refers to a recombinant nucleic acid molecule comprising an expression control sequence operatively linked with a heterologous nucleic acid sequence. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences). As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

As used herein, a “recombinant cell” refers to a cell comprising a recombinant nucleic acid molecule, e.g., a cell comprising an expression construct.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

As used herein, an amino acid sequence “consists of” only the amino acids in that sequence.

As used herein, a chemical entity, such as a polypeptide, is “substantially pure” if it is the predominant chemical entity of its kind (e.g., of polypeptides) in a composition. This includes the chemical entity representing more than 50%, more than 80%, more than 90%, more than 95%, more than 98%, more than 99%, more than 99.5%, more than 99.9%, or more than 99.99% of the chemical entities of its kind in the composition.

The antibodies and biding-fragments thereof can be “a conjugated antibody” or a “non-conjugated antibody” (that is, not conjugated). The term “conjugate” refers to a first molecule, e.g., an antibody (an “immunoconjugate”), chemically coupled with a moiety, such as a detectable label or a biologically active moiety, such as a drug, toxin or chemotherapeutic or cytotoxic agent. Accordingly, this disclosure contemplates antibodies conjugated with one or more moieties.

As used herein, the term “labeled” molecule refers to a molecule that is bound to a detectable label, either covalently, through a linker or a chemical bond, or noncovalently, through ionic, van der Waals, electrostatic, or hydrogen bonds, such that the presence of the molecule may be detected by detecting the presence of the detectable label bound to the molecule.

As used herein, the term “detectable label” refers to a composition detectable by spectroscopic, photochemical, biochemical, immunochemical, chemical, or other physical means. Examples of detectable labels are described herein and include, without limitation, colorimetric, fluorescent, chemiluminescent, enzymatic, and radioactive labels. For the purposes of the present disclosure, a detectable label can also be a moiety that does not itself produce a signal (e.g., biotin), but that binds to a second moiety that is able to produce a signal (e.g., labeled avidin).

### II. Antibodies

The present invention provides antibodies and antigen-binding fragments thereof that bind SARS-CoV-2 spike protein. The antibodies of the present invention are useful, inter alia, for inhibiting or neutralizing the activity of COVID-19 spike protein. In some embodiments, the antibodies are useful for blocking binding of the virus to its host cell receptor angiotensin converting enzyme 2 (ACE2) and for preventing the entry of COVID-19 virus into host cells. In some embodiments, the antibodies function by inhibiting the cell-to-cell transmission of the virus. In certain embodiments, the antibodies are useful in preventing, treating or ameliorating at least one symptom of SARS-CoV-2 infection in a subject. In certain embodiments, the antibodies may be administered prophylactically or therapeutically to a subject having or at risk of having SARS-CoV-2 infection.

The antibodies of the invention can be full-length (e.g., “intact”) (for example, an IgG1 or IgG4 antibody) or may comprise only an antigen-binding portion (for example, a Fab, F(ab)2 or scFv fragment), and may be modified to affect functionality, e.g., to increase persistence in the host or to eliminate residual effector functions (Reddy et al., 2000, J. Immunol. 164:1925-1933). In certain embodiments, the antibodies may be bispecific.

In a first aspect, the present invention provides isolated recombinant monoclonal antibodies or antigen-binding fragments thereof that bind specifically to the SARS-CoV-2 spike protein. In some embodiments, the antibodies are fully human monoclonal antibodies. The antibodies and antigen-binding fragments thereof of the invention bind to an epitope within the receptor binding domain (RBD) of the spike protein of SARS-CoV-2. In some embodiments, the present invention provides antibodies and antigen-binding fragments thereof that bind to spike protein of different SARS-CoV-2 isolates.

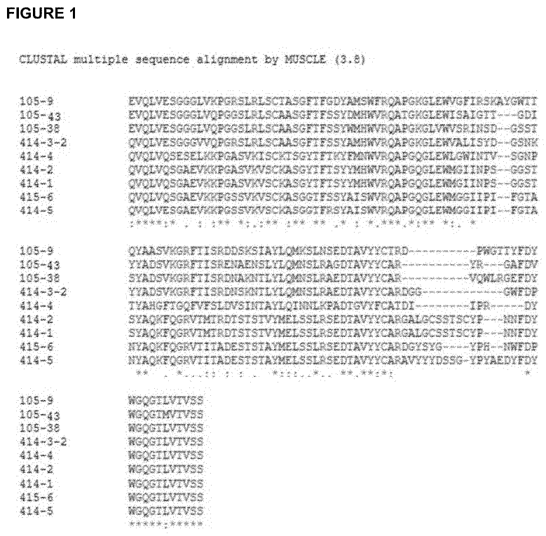

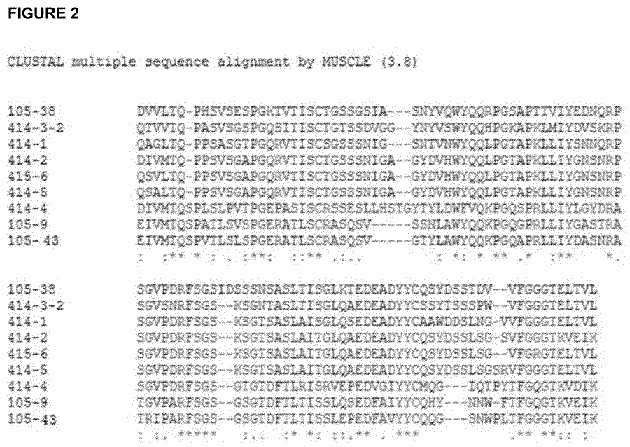

Exemplary anti-COVID-19 spike antibodies of the present invention are listed in FIGS. 1 and 2 herein. FIG. 1 sets forth the amino acid sequence identifiers of the heavy chain variable regions (HCVRs), and heavy chain complementarity determining regions (HCDRI, HCDR2 and HCDR3); FIG. 2 sets forth the amino acid sequence identifiers of the light chain variable regions (LCVRs), and light chain complementarity determining regions (LCDRI, LCDR2 and LCDR3) of exemplary anti-COVID-19 antibodies. SEQ NOs 1-9 and 19-27 set forth the nucleic acid sequences of the HCVRs, LCVRs, HCDRI, HCDR2 HCDR3, LCDRI, LCDR2 and LCDR3 of the exemplary anti-COVID-19 antibodies. SEQ NOs 10-18 and 28-36 set forth the nucleic acid sequences of the HCVRs, LCVRs, HCDRI, HCDR2 HCDR3, LCDRI, LCDR2 and LCDR3 of the exemplary anti-COVID-19 antibodies.

The present invention provides antibodies, or antigen-binding fragments thereof, comprising an HCVR comprising an amino acid sequence selected from any of the HCVR amino acid sequences listed in FIG. 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in FIG. 2, or a substantially similar sequence thereof having at least 90%, at least 95%, at least 98% or at least 99% sequence identity thereto. The present invention also provides antibodies, or antigen-binding fragments thereof, comprising an HCVR and an LCVR amino acid sequence pair (HCVR/LCVR) comprising any of the HCVR amino acid sequences listed in FIG. 1 paired with any of the LCVR amino acid sequences listed in FIG. 2. According to certain embodiments, the present invention provides antibodies, or antigen-binding fragments thereof, comprising an HCVR/LCVR amino acid sequence pair contained within any of the exemplary anti-COVID-19 antibodies listed in FIGS. 1 and 2. In certain embodiments, the HCVR/LCVR amino acid sequence pair is selected from the group consisting of SEQ ID NOs: 10/28, 11/29, 12/30, 13/31, 14/32, 15/33, 16/34, 17/35, 18/36.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Martin, “Protein Sequence and Structure Analysis of Antibody Variable Domains,” Antibody Engineering Vol. 2, DOI 10.1007/978-3-642-01147-4_3. Public databases are also available for identifying CDR sequences within an antibody.

The present invention includes anti-COVID-19 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC). The present invention also provides for antibodies and antigen-binding fragments thereof that compete for specific binding to SARS-CoV-2-S with an antibody or antigen-binding fragment thereof comprising the CDRs of a HCVR and the CDRs of a LCVR, wherein the HCVR and LCVR each has an amino acid sequence selected from the HCVR and LCVR sequences listed in FIGS. 1 and 2.

The present invention provides isolated antibodies and antigen-binding fragments thereof that block COVID-19 spike protein binding to ACE2. In some embodiments, the antibody or antigen-binding fragment thereof that blocks SARS-CoV-2 spike protein binding to ACE2 may bind to the same epitope on SARS-CoV-2 spike protein as ACE2 or may bind to a different epitope on SARS-CoV-2 spike protein as ACE2. In some embodiments, the present invention provides antibodies or antigen-binding fragments thereof that block the binding of SARS-CoV-2 to human, pangolin and bat ACE2.

In certain embodiments, the antibodies or antigen-binding fragments of the present invention are bispecific comprising a first binding specificity to a first epitope in the receptor binding domain of SARS-CoV-2 spike protein and a second binding specificity to a second epitope in the receptor binding domain of SARS-CoV-2 spike protein wherein the first and second epitopes are distinct and non-overlapping. In one embodiment, the invention provides an isolated antibody or antigen-binding fragment that has one or more of the following characteristics: (a) is a fully human monoclonal antibody; (b) is a fully recombinant human antibody produced by transfection or stable integration of the antibody coding sequences in an appropriate expression host such as 293T or Chinese hamster ovary cell lines; (c) interacts with one or more amino acid residues in the receptor binding domain of SARS-CoV-2 spike protein selected from amino acid residues 331-524 of SEQ ID NO: 41; (d) blocks binding of SARS-CoV-2 spike protein to angiotensin converting enzyme 2 (ACE2) by more than 90%, as measured in a blocking ELISA assay; (e) neutralizes SARS-CoV-2 infectivity of human host cells by more than 90% and with an IC50 less than 25 nM, as measured in a virus-like particle (VLP) neutralization assay; (f) neutralizes SARS-CoV-2 infectivity wherein the SARS-CoV-2 comprises an isolate of the virus derived from infected individuals; (g) is a bi-specific antibody comprising a first binding specificity to a first epitope in the receptor binding domain of COVID-19 spike protein and a second binding specificity to a second epitope in the receptor binding domain of COVID-19 spike protein wherein the first and second epitopes are distinct and non-overlapping.

### III. Nucleic Acid Molecules and Recombinant Cells

In a second aspect, the present invention provides nucleic acid molecules encoding anti-SARS-CoV-2 spike antibodies or portions thereof. For example, the present invention provides nucleic acid molecules encoding any of the HCVR amino acid sequences listed in FIG. 1; in certain embodiments the nucleic acid molecule comprises a polynucleotide sequence selected from any of the HCVR nucleic acid sequences listed in SEQ ID NO. 1-9, or a substantially similar sequence thereof having at least 90%, at least 95%, at least 98% or at least 99% sequence identity thereto.

The present invention also provides nucleic acid molecules encoding any of the LCVR amino acid sequences listed in FIG. 2; in certain embodiments the nucleic acid molecule comprises a polynucleotide sequence selected from any of the LCVR nucleic acid sequences listed in SEQ ID NO. 19-27, or a substantially similar sequence thereof having at least 90%, at least 95%, at least 98% or at least 99% sequence identity thereto.

The present invention also provides nucleic acid molecules encoding any of the HCDRI amino acid sequences listed in FIG. 1; in certain embodiments the nucleic acid molecule comprises a polynucleotide sequence selected from any of the HCDRI nucleic acid sequences listed in SEQ ID NO. 1-9, or a substantially similar sequence thereof having at least 90%, at least 95%, at least 98% or at least 99% sequence identity thereto.

The present invention also provides nucleic acid molecules encoding any of the HCDR2 amino acid sequences listed in FIG. 1; in certain embodiments the nucleic acid molecule comprises a polynucleotide sequence selected from any of the HCDR2 nucleic acid sequences listed in SEQ ID NO. 1-9, or a substantially similar sequence thereof having at least 90%, at least 95%, at least 98% or at least 99% sequence identity thereto.

The present invention also provides nucleic acid molecules encoding any of the HCDR3 amino acid sequences listed in FIG. 1; in certain embodiments the nucleic acid molecule comprises a polynucleotide sequence selected from any of the HCDR3 nucleic acid sequences listed in SEQ ID NO. 1-9, or a substantially similar sequence thereof having at least 90%, at least 95%, at least 98% or at least 99% sequence identity thereto.

The present invention also provides nucleic acid molecules encoding any of the LCDRI amino acid sequences listed in FIG. 2; in certain embodiments the nucleic acid molecule comprises a polynucleotide sequence selected from any of the LCDRI nucleic acid sequences listed in SEQ ID NO. 19-27, or a substantially similar sequence thereof having at least 90%, at least 95%, at least 98% or at least 99% sequence identity thereto.

The present invention also provides nucleic acid molecules encoding any of the LCDR2 amino acid sequences listed in FIG. 2; in certain embodiments the nucleic acid molecule comprises a polynucleotide sequence selected from any of the LCDR2 nucleic acid sequences listed in SEQ ID NO. 19-27, or a substantially similar sequence thereof having at least 90%, at least 95%, at least 98% or at least 99% sequence identity thereto.

The present invention also provides nucleic acid molecules encoding any of the LCDR3 amino acid sequences listed in FIG. 2; in certain embodiments the nucleic acid molecule comprises a polynucleotide sequence selected from any of the LCDR3 nucleic acid sequences listed in SEQ ID NO. 19-27, or a substantially similar sequence thereof having at least 90%, at least 95%, at least 98% or at least 99% sequence identity thereto.

The present invention also provides nucleic acid molecules encoding a human heavy chain constant region and a light chain constant region with which any of the HCVRs and LCVRs of FIGS. 1 and 2 may be paired to form a functional anti-COVID-19 antibody. The nucleotide sequences for the heavy chain constant region are given in SEQ ID NO. 37 and the corresponding amino acid sequence in SEQ ID NO. 38. The nucleotide sequences for the light chain constant region are given in SEQ ID NO. 39 and the corresponding amino acid sequence in SEQ ID NO. 40.

The present invention provides nucleic acid molecules encoding any of the heavy chain amino acid sequences listed in FIG. 1. The present invention also provides nucleic acid molecules encoding any of the light chain amino acid sequences listed in FIG. 2.

In a related aspect, the present invention provides recombinant expression vectors capable of expressing a polypeptide comprising a heavy or light chain variable region of an anti-SARS-CoV-2 antibody. For example, the present invention includes recombinant expression vectors comprising any of the nucleic acid molecules mentioned above, i.e., nucleic acid molecules encoding any of the HCVR, LCVR, and/or CDR sequences as set forth in FIGS. 1 and 2. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

In another aspect, provided herein is a recombinant nucleic acid molecule encoding an antibody or antigen-binding fragment of an antibody that binds to a SARS-CoV-2 spike protein, as provided herein. In one embodiment the nucleic acid molecule comprises nucleotide sequences encoding: three heavy chain complementarity determining regions (CDRs) (HCDRI, HCDR2 and HCDR3) contained within any one of the heavy chain variable region (HCVR) sequences selected from the group consisting of SEQ ID NOs: 10-18; and three light chain CDRs (LCDRI, LCDR2 and LCDR3) contained within any one of the light chain variable region (LCVR) sequences selected from the group consisting of SEQ ID NOs: 28-36, wherein the antibody or antigen-binding fragment comprises a HCVR/LCVR amino acid sequence pair selected from the group consisting of SEQ ID NOs: 10/28, 11/29, 12/30, 13/31, 14/32, 15/33, 16/34, 17/35, 18/36. In another embodiment the nucleic acid molecule comprises a nucleotide sequence selected from any of SEQ ID NOs: 1-9 and SEQ ID NOs: 19-27. a pair of nucleotide sequences selected from SEQ ID NOs: 1/19, 2/20, 3/21, 4/22, 5/23, 6/24, 7/25, and 8/26.

In another aspect, provided herein is a recombinant expression construct comprising an expression control sequence operatively linked to a recombinant nucleic acid molecule encoding an antibody or antigen biding fragment of an antibody that binds to a SARS-CoV-2 spike protein, as provided herein.

In another aspect, provided herein is a recombinant host cell comprising a recombinant expression construct comprising an expression control sequence operatively linked to a recombinant nucleic acid molecule encoding an antibody or antigen biding fragment of an antibody that binds to a SARS-CoV-2 spike protein, as provided herein.

### IV. Pharmaceutical Compositions

In a third aspect, the invention provides a pharmaceutical composition comprising a therapeutically effective amount of at least one recombinant monoclonal antibody or antigen-binding fragment thereof which specifically binds COVID-19 spike protein and a pharmaceutically acceptable carrier. In a related aspect, the invention features a composition which is a combination of an anti-SARS-CoV-2-S antibody and a second therapeutic agent. In one embodiment, the second therapeutic agent is any agent that is advantageously combined with an anti-COVID-19 antibody. Exemplary agents that may be advantageously combined with an anti-SARS-CoV-2 antibody include, without limitation, other agents that bind and/or inhibit SARS-CoV-2 activity (including other antibodies or antigen-binding fragments thereof, etc.) and/or agents which do not directly bind SARS-CoV-2-S but nonetheless inhibit viral activity including infectivity of host cells. In certain embodiments, the invention provides for a pharmaceutical composition comprising: (a) a first anti-SARS-CoV-2-S antibody or antigen-binding fragment thereof; (b) a second anti-SARS-CoV-2-S antibody or antigen-binding fragment thereof, wherein the first antibody binds to a first epitope on SARS-CoV-2 spike protein and the second antibody binds to a second epitope on SARS-CoV-2 spike protein wherein the first and second epitopes are distinct and non-overlapping; and (c) a pharmaceutically acceptable carrier or diluent. In certain embodiments, the invention provides for a pharmaceutical composition comprising: (a) a first anti-SARS-CoV-2-S antibody or antigen-binding fragment thereof; (b) a second anti-SARS-CoV-2-S antibody or antigen-binding fragment thereof, wherein the first antibody does not cross-compete with the second antibody for binding to SARS-CoV-2 spike protein; and (c) a pharmaceutically acceptable carrier or diluent.

### V. Methods of Diagnosis

In another aspect, provided herein is a method of detecting SARS-CoV-2 comprising performing an immunoassay on a biological sample from a subject; wherein the immunoassay uses an antibody or antigen-binding fragment of an antibody that binds to a SARS-CoV-2 spike protein, as provided herein. In one embodiment the immunoassay selected from radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS). In another embodiment the subject is a human or a nonhuman mammal, e.g., pangolin, a bat, a civet or a camel.

In another aspect, provided herein is the use of an antibody or antigen-binding fragment as described herein in the preparation of a diagnostic for the detection of SARS-CoV-2 in a sample.

### VI. Methods of Treatment

In another aspect, the invention provides therapeutic methods for treating a disease or disorder associated with SARS-CoV-2 such as viral infection in a subject using an anti-SARS-CoV-2-S antibody or antigen-binding portion of an antibody of the invention, wherein the therapeutic methods comprise administering a therapeutically effective amount of a pharmaceutical composition comprising an antibody or antigen-binding fragment of an antibody of the invention to the subject in need thereof. The disorder treated is any disease or condition which is improved, ameliorated, inhibited or prevented by inhibition of SARS-CoV-2 activity. In certain embodiments, the invention provides methods to prevent, treat or ameliorate at least one symptom of COVID-19 infection, the method comprising administering a therapeutically effective amount of an anti-SARS-CoV-2-S antibody or antigen-binding fragment thereof of the invention to a subject in need thereof. In some embodiments, the present invention provides methods to ameliorate or reduce the severity of at least one symptom or indication of COVID-19 infection in a subject by administering an anti-COVID-19 antibody of the invention, wherein the at least one symptom or indication is selected from the group consisting of inflammation in the lung, alveolar damage, fever, cough, shortness of breath, diarrhea, organ failure, pneumonia, septic shock and death. In certain embodiments, the invention provides methods to decrease viral load in a subject, the methods comprising administering to the subject an effective amount of an antibody or fragment thereof of the invention that binds SARS-CoV-2-S and blocks SARS-CoV-2 binding to host cell receptor ACE2. In some embodiments, the antibody or antigen-binding fragment thereof may be administered prophylactically or therapeutically to a subject having or at risk of having COVID-19 infection. The subjects at risk include, but are not limited to, an immunocompromised person, an elderly adult (more than 65 years of age), children younger than 2 years of age, travelers to or from any country with known incidence to COVID-19 infection, healthcare workers, adults or children in close contact with a person(s) with confirmed or suspected COVID-19 infection, and people with underlying medical conditions such as pulmonary infection, heart disease or diabetes. In certain embodiments, the antibody or antigen-binding fragment thereof the invention is administered in combination with a second therapeutic agent to the subject in need thereof. The second therapeutic agent may be selected from the group consisting of any non-contraindicated anti-inflammatory drug (such as acetaminophen), an anti-infective drug, a different antibody to SARS-CoV-2 spike protein, an anti-viral drug, a vaccine for SARS-CoV-2, a dietary supplement such as anti-oxidants and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur. The antibody or fragment thereof may be administered subcutaneously, intravenously, intradermally, intraperitoneally, orally, intra-muscularly, or intracranially. In one embodiment, the anti-body may be administered as a single intravenous infusion for maximum concentration of the antibody in the serum of the subject. The antibody or fragment thereof may be administered at a dose of about 0.1 mg/kg of body weight to about 100 mg/kg of body weight of the subject. In certain embodiments, an antibody of the present invention may be administered at one or more doses comprising between 20 mg to 600 mg.

The present invention also includes use of an anti-COVID-19 antibody or antigen-binding fragment thereof of the invention in the manufacture of a medicament for the treatment of a disease or disorder that would benefit from the blockade of SARS-CoV-2 binding and/or activity.

In another aspect, provided herein is a method of treating a subject infected with SARS-CoV-2 comprising administering to the subject an effective amount of a pharmaceutical composition comprising an antibody or antigen-binding fragment of an antibody that binds to a SARS-CoV-2 spike protein, as provided herein and a pharmaceutically acceptable carrier or diluent.

In another aspect, provided herein is a method of treating a subject infected with SARS-CoV-2 comprising performing plasmapheresis on the subject using plasma filter comprising a microporous fiber having immobilized thereto an antibody or antigen-binding fragment of an antibody that binds to a SARS-CoV-2 spike protein, as provided herein wherein the antibody or the antigen-binding fragment of the antibody removes SARS-CoV-2 from the blood of the subject.

### VII. Kits and Articles

In a sixth aspect, the present invention includes use of anti-COVID-19 antibody or antigen-binding fragment thereof of the invention in the manufacture of in vitro diagnostics for the rapid detection of COVID-19 virus and/or portions thereof in body fluids, tissues, secretions, and waste products derived from humans or potential COVID-19 animal reservoirs, including but not limited to pangolins, bats, and other known animal reservoirs (e.g., civets and dromedary camels) of the genetically related betacoronaviruses referred to as SARS-CoV and MERS. These body fluids, tissues, secretions, and waste products include, but are not limited to, blood, plasma, serum, tears, saliva, cerebrospinal fluid, amniotic fluid, exhaled breath condensate, urine and feces.

In a seventh aspect, the present invention includes use of anti-COVID-19 antibody or antigen-binding fragment thereof of the invention in the application and manufacture of solid supports/matrices employed in plasmapheresis to affect the concentration of COVID-19 viral particles and/or fragments derived therefrom. Such antibody-bearing scaffolds may have utility in active removal of COVID-19 particles from patient or donor plasma, thereby decreasing viral load.

In another aspect provided herein is a solid support bearing one or more antibody or antigen-binding fragments that bind to SARS-CoV-2 spike protein as provided herein. In one embodiment, the support comprises a microporous filter, e.g., a hollow microfiber configured for use in plasmapheresis.

In another aspect, provided herein is a filter device comprising an elongated, hollow tube comprising and entrance and an exit and, within the tube, a plurality of elongated, microporous fibers having an interior lumen extending along the length thereof, wherein the microporous fibers comprise, immobilized thereto, an antibody or antigen binding fragment that binds to a SARS-CoV-2 spike protein, as provided herein.

### VIII. Methods of Making

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

Binding fragments of antibodies can be produced by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology.

Methods for humanizing non-human antibodies (i.e., using CDRs from non-human antibodies) are also known in the art. Generally, a humanized antibody has one or more amino acid residues from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments.

All publications, patents, and patent applications mentioned in this specification are herein incorporated by reference to the same extent as if each individual publication, patent, or patent application was specifically and individually indicated to be incorporated by reference.

### REFERENCES CITED

Ge, X.-Y., Li, J.-L., Yang, X.-L., Chmura, A. A., Zhu, G., Epstein, J. H., Mazet, J. K., Hu, B., Zhang, W., Peng, C., et al. (2013). Isolation and characterization of a bat SARS-like coronavirus that uses the ACE2 receptor. Nature 503, 535-538.
Luan, J., Lu, Y., Jin, X., and Zhang, L. (2020). Spike protein recognition of mammalian ACE2 predicts the host range and an optimized ACE2 for SARS-CoV-2 infection. Biochem. Biophys. Res. Commun.
Ortega, J. T., Serrano, M. L., Pujol, F. H., and Rangel, H. R. (2020). Role of changes in SARS-CoV-2 spike protein in the interaction with the human ACE2 receptor: An in silico analysis. EXCLI J. 19, 410-417.
Xu, J., Jia, W., Wang, P., Zhang, S., Shi, X., Wang, X., and Zhang, L. (2019). Antibodies and vaccines against Middle East respiratory syndrome coronavirus. Emerg. Microbes Infect. 8, 841-856.
Zhang, T., Wu, Q., and Zhang, Z. (2020). Probable Pangolin Origin of SARS-CoV-2 Associated with the COVID-19 Outbreak. Curr. Biol. CB.

SEQUENCE LISTING
Clone catalog no. AM002414
Original Clone ID: 414-2 HCVR1
Sequence Length: 378 bases
SEQ ID NO. 1
CAGGTGCAGCTGGTGCAGTCTGGGGCTGAGGTGAAGAAGCCTGGGGCCTC
AGTGAAGGTTTCCTGCAAGGCATCTGGATACACCTTCACCAGCTACTATA
TGCACTGGGTGCGACAGGCCCCTGGACAAGGGCTTGAGTGGATGGGAATA
ATCAACCCTAGTGGTGGTAGCACAAGCTACGCACAGAAGTTCCAGGGCAG
AGTCACCATGACCAGGGACACGTCCACGAGCACAGTCTACATGGAGCTGA
GCAGCCTGAGATCTGAGGACACGGCCGTGTATTACTGTGCGAGAGGGGCC
CTGGGGTGTAGTAGTACCAGCTGCTATCCTAACAACTTTGACTACTGGGG
CCAGGGAACCCTGGTCACCGTCTCCTCA
Clone catalog no. AM038105
Original Clone ID: 105-38 HCVR2
Sequence Length: 357 bases
SEQ ID NO. 2
GAGGTGCAGCTGGTGGAGTCCGGGGGAGGCTTAGTTCAGCCTGGGGGGTC
CCTGAGACTCTCCTGTGCAGCCTCTGGATTCACCTTCAGTAGCTACTGGA
TGCACTGGGTCCGCCAAGCTCCAGGGAAGGGGCTGGTGTGGGTCTCACGT
ATTAATAGTGATGGGAGTAGCACAAGCTACGCGGACTCCGTGAAGGGCCG
ATTCACCATCTCCAGAGACAACGCCAAGAACACGCTGTATCTGCAAATGA
ACAGTCTGAGAGCCGAGGACACGGCTGTGTATTACTGTGCAAGAGTGCAG
TGGCTACGGGGGGAATTTGACTACTGGGGCCAGGGAACCCTGGTCACCGT
CTCCTCA
Clone catalog no. AM009105
Original Clone ID: 105-9 HCVR3
Sequence Length: 363 bases
SEQ ID NO. 3
GAAGTGCAGCTGGTGGAGTCTGGGGGAGGCTTGGTAAAGCCAGGGCGGTC
CCTGAGACTCTCCTGTACAGCTTCTGGATTCACCTTTGGTGATTATGCTA
TGAGCTGGTTCCGCCAGGCTCCAGGGAAGGGGCTGGAGTGGGTAGGTTTC
ATTAGAAGCAAAGCATATGGTTGGACAACACAATATGCCGCGTCTGTGAA
AGGCAGATTCACCATCTCAAGAGATGATTCCAAAAGCATCGCCTATCTGC
AAATGAAGAGCCTGAACAGCGAGGACACAGCCGTGTATTACTGTACTAGA
GATCCATGGGGCACTACGTACTTTGACTACTGGGGCCAGGGAACCCTGGT
CACCGTCTCCTCA
Clone catalog no. AM006415
Original Clone ID: 415-6 HCVR4
Sequence Length: 369 bases
SEQ ID NO. 4
CAGGTGCAGCTGGTGCAATCTGGGGCTGAGGTGAAGAAGCCTGGGTCCTC
GGTGAAGGTCTCCTGCAAGGCTTCTGGAGGCACCTTCAGCAGCTATGCTA
TCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAG
AGTCACGATTACCGCGGACGAATCCACGAGCACAGCCTACATGGAGCTGA
GCAGCCTGAGATCTGAGGACACGGCCGTGTATTACTGTGCGAGAGATGGA
TACAGCTATGGTTATCCCCACAACTGGTTCGACCCCTGGGGCCAGGGAAC
CCTGGTCACCGTCTCCTCA
Clone catalog no. AM001414
Original Clone ID: 414-1 HCVR5
Sequence Length: 378 bases
SEQ ID NO. 5
CAGGTGCAGCTGGTGCAGTCTGGGGCTGAGGTGAAGAAGCCTGGGGCCTC
AGTGAAGGTTTCCTGCAAGGCATCTGGATACACCTTCACCAGCTACTATA
TGCACTGGGTGCGACAGGCCCCTGGACAAGGGCTTGAGTGGATGGGAATA
ATCAACCCTAGTGGTGGTAGCACAAGCTACGCACAGAAGTTCCAGGGCAG
AGTCACCATGACCAGGGACACGTCCACGAGCACAGTCTACATGGAGCTGA
GCAGCCTGAGATCTGAGGACACGGCCGTGTATTACTGTGCGAGAGGGGCC
CTGGGGTGTAGTAGTACCAGCTGCTATCCTAACAACTTTGACTACTGGGG
CCAGGGAACCCTGGTCACCGTCTCCTCA
Clone catalog no. AM043105
Original Clone ID: 105-43 HCVR6
Sequence Length: 345 bases
SEQ ID NO. 6
GAAGTGCAGCTGGTGGAGTCTGGGGGAGGCTTGGTACAGCCGGGGGGGTC
CCTGAGACTCTCCTGTGCAGCCTCGGGATTCACCTTCAGTAGTTACGACA
TGCACTGGGTCCGCCAAGCTACAGGAAAAGGTCTGGAATGGATCTCAGCT
ATTGGTACTACTGGTGACATATACTATGCAGACTCCGTGAAGGGCCGATT
CACCATCTCCAGAGAAAATGCCGAGAACTCCTTGTATCTTCAAATGAACA
GCCTGAGAGCCGGGGACACGGCTGTGTATTACTGTGCAAGATATAGGGGT
GCTTTTGATGTCTGGGGCCAAGGGACAATGGTCACCGTCTCTTCA
Clone catalog no. AM043105
Original Clone ID: 414-4 HCVR7
Sequence Length: 348 bases
SEQ ID NO. 7
CAGGTGCAGCTGGTGCAATCTGAGTCTGAGTTGAAGAAGCCTGGGGCCTC
AGTGAAGATTTCCTGCAAGACTTCTGGATACACCTTCACTAAATACTTTA
TGAATTGGGTGCGACAGGCCCCTGGACAAGGGCTTGAGTGGCTGGGATGG
ATTAACACCGTCAGTGGGAACCCAACCTATGCCCACGGCTTCACAGGGCA
GTTTGTCTTCTCCTTGGACGTCTCTATCAACACGGCATATCTTCAGATCA
ACAACCTAAAGCCTGCGGACACTGGCGTCTATTTCTGTGCGACAGATATT
ATTCCGAGGGACTACTGGGGCCAGGGAACCCTGGTCACCGTCTCCTCA
Clone catalog no. AM005415
Original Clone ID: 415-5 HCVR8
Sequence Length: 384 bases
SEQ ID NO. 8
CAGGTGCAGCTGGTGGAGTCTGGGGCTGAGGTGAAGAAGCCTGGGTCCTC
GGTGAAGGTCTCCTGCAAGGCTTCTGGAGGCACCTTCAGGAGCTATGCTA
TCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAG
AGTCACGATTACCGCGGACGAATCCACGAGCACAGCCTACATGGAGCTGA
GCAGCCTGAGATCTGAGGACACGGCCGTGTATTACTGTGCGAGGGCCGTG
TATTACTATGATAGTAGTGGGTACCCATACGCCGAAGACTACTTTGACTA
CTGGGGCCAGGGAACCCTGGTCACCGTCTCCTCA
Clone catalog no. AM032414
Original Clone ID: 414-3-2 HCVR9
Sequence Length: 351 bases
SEQ ID NO. 9
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAGCTATGCTA
TGCACTGGGTCCGCCAGGCTCCAGGCAAGGGGCTGGAGTGGGTGGCACTT
ATATCATATGATGGAAGTAATAAATACTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAGAGACGGC
GGGGGCTGGTTCGACCCCTGGGGCCAGGGAACCCTGGTCACCGTCTCCTC
A
Clone catalog no. AM002414
Original Clone ID: 414-2 HCVR1
SEQ ID NO. 10
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARGA
LGCSSTSCYPNNFDYWGQGTLVTVSS
Clone catalog no. AM038105
Original Clone ID: 105-38 HCVR2
SEQ ID NO. 11
EVQLVESGGGLVQPGGSLRLSCAASGFTFSSYWMHWVRQAPGKGLVWVSR
INSDGSSTSYADSVKGRFTISRDNAKNTLYLQMNSLRAEDTAVYYCARVQ
WLRGEFDYWGQGTLVTVSS
Clone catalog no. AM009105
Original Clone ID: 105-9 HCVR3
SEQ ID NO. 12
EVQLVESGGGLVKPGRSLRLSCTASGFTFGDYAMSWFRQAPGKGLEVVVG
FIRSKAYGVVTTQYAASVKGRFTISRDDSKSIAYLQMKSLNSEDTAVYYC
TRDPWGTTYFDYWGQGTLVTVSS
Clone catalog no. AM006415
Original Clone ID: 415-6 HCVR4
SEQ ID NO. 13
QVQLVQSGAEVKKPGSSVKVSCKASGGTFSSYAISVVVRQAPGQGLEWMG
GIIPIFGTANYAQKFQGRVTITADESTSTAYMELSSLRSEDTAVYYCARD
GYSYGYPHNWFDPWGQGTLVTVSS
Clone catalog no. AM001414
Original Clone ID: 414-1 HCVR5
SEQ ID NO. 14
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARGA
LGCSSTSCYPNNFDYWGQGTLVTVSS
Clone catalog no. AM043105
Original Clone ID: 105-4 HCVR6
SEQ ID NO. 15
EVQLVESGGGLVQPGGSLRLSCAASGFTFSSYDMHVVVRQATGKGLEWIS
AIGTTGDIYYADSVKGRFTISRENAENSLYLQMNSLRAGDTAVYYCARYR
GAFDVWGQGTMVTVSS
Clone catalog no. AM043105
Original Clone ID: 414-4 HCVR7
SEQ ID NO. 16
QVQLVQSESELKKPGASVKISCKTSGYTFTKYFMNVVVRQAPGQGLEWLG
WINTVSGNPTYAHGFTGQFVFSLDVSINTAYLQINNLKPADTGVYFCATD
IIPRDYWGQGTLVTVSS
Clone catalog no. AM005415
Original Clone ID: 415-5 HCVR8
SEQ ID NO. 17
QVQLVESGAEVKKPGSSVKVSCKASGGTFRSYAISWVRQAPGQGLEWMGG
IIPIFGTANYAQKFQGRVTITADESTSTAYMELSSLRSEDTAVYYCARAV
YYYDSSGYPYAEDYFDYWGQGTLVTVSS
Clone catalog no. AM032414
Original Clone ID: 414-3-2 HCVR9
SEQ ID NO. 18
QVQLVESGGGVVQPGRSLRLSCAASGFTFSSYAMHVVVRQAPGKGLEWVA
LISYDGSNKYYADSVKGRFTISRDNSKNTLYLQMNSLRAEDTAVYYCARD
GGGWFDPWGQGTLVTVSS
Clone catalog no. AM002414
Original Clone ID: 414-2 LCVR1
Sequence Length: 333 bases
SEQ ID NO. 19
GATATTGTGATGACTCAGCCGCCCTCAGTGTCTGGGGCCCCAGGGCAGAG
GGTCACCATCTCCTGCACTGGGAGCAGCTCCAACATCGGGGCAGGTTATG
ATGTACACTGGTACCAGCAGCTTCCAGGAACAGCCCCCAAACTCCTCATC
TATGGTAACAGCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGGTTCG
GTGTTCGGCGGAGGGACCAAGGTGGAAATCAAA
Clone catalog no. AM038105
Original Clone ID: 105-38 LCVR2
Sequence Length: 333 bases
SEQ ID NO. 20
GATGTTGTGCTGACACAGCCCCACTCTGTGTCGGAGTCTCCGGGGAAGAC
GGTAACCATCTCCTGCACCGGCAGCAGTGGCAGCATTGCCAGCAACTATG
TGCAGTGGTACCAGCAGCGCCCGGGCAGTGCCCCCACCACTGTGATCTAT
GAGGATAACCAAAGACCCTCTGGGGTCCCTGATCGGTTCTCTGGCTCCAT
CGACAGCTCCTCCAACTCTGCCTCCCTCACCATCTCTGGACTGAAGACTG
AGGACGAGGCTGACTACTACTGTCAGTCTTATGATAGCAGCACCGATGTG
GTATTCGGCGGAGGGACCGAGCTGACCGTCCTA
Clone catalog no. AM009105
Original Clone ID: 105-9 LCVR3
Sequence Length: 318 bases
SEQ ID NO. 21
GAAATAGTGATGACGCAGTCTCCAGCCACCCTGTCTGTGTCTCCAGGGGA
AAGAGCCACCCTCTCCTGCAGGGCCAGTCAGAGTGTTAGCAGCAACTTAG
CCTGGTACCAGCAGAAACCTGGCCAGGGTCCCAGGCTCCTCATCTATGGT
GCATCCACCAGGGCCACTGGTGTCCCAGCCAGGTTCAGTGGCAGTGGGTC
TGGGACAGACTTCACTCTCACCATCAGCAGCCTGCAGTCTGAAGATTTTG
CAATTTATTACTGTCAGCACTATAATAACTGGTTCACTTTTGGCCAGGGG
ACCAAGGTGGAGATCAAA
Clone catalog no. AM006415
Original Clone ID: 415-6 LCVR4
Sequence Length: 330 bases
SEQ ID NO. 22
CAGTCTGTGCTGACTCAGCCGCCCTCAGTGTCTGGGGCCCCAGGGCAGAG
GGTCACCATCTCCTGCACTGGGAGCAGCTCCAACATCGGGGCAGGTTATG
ATGTACACTGGTACCAGCAGCTTCCAGGAACAGCCCCCAAACTCCTCATC
TATGGTAACAGCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGGGGTG
TTCGGCAGAGGGACCGAGCTGACCGTCCTA
Clone catalog no. AM001414
Original Clone ID: 414-1 LCVR5
Sequence Length: 330 bases
SEQ ID NO. 23
CAGGCAGGGCTGACTCAGCCACCCTCAGCGTCTGGGACCCCCGGGCAGAG
GGTCACCATCTCTTGTTCTGGAAGCAGCTCCAACATCGGAAGTAATACTG
TAAACTGGTACCAGCAGCTCCCAGGAACGGCCCCCAAACTCCTCATCTAT
AGTAATAATCAGCGGCCCTCAGGGGTCCCTGACCGATTCTCTGGCTCCAA
GTCTGGCACCTCAGCCTCCCTGGCCATCAGTGGGCTCCAGTCTGAGGATG
AGGCTGATTATTACTGTGCAGCATGGGATGACAGCCTGAATGGTGTGGTA
TTCGGCGGAGGGACCGAGCTGACCGTCCTA
Clone catalog no. AM043105
Original Clone ID: 105-43 LCVR6
Sequence Length: 321 bases
SEQ ID NO. 24
GAAATAGTGATGACGCAGTCTCCAGTCACCCTGTCTTTGTCTCCAGGGGA
AAGAGCCACCCTCTCCTGCAGGGCCAGTCAGAGTGTTGGCACCTACTTAG
CCTGGTACCAACAGAAACCTGGCCAGGCTCCCAGGCTCCTCATCTATGAT
GCATCCAACAGGGCCACTCGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGGGTAGCAACTGGCCGCTCACTTTCGGCGGA
GGGACCAAGGTGGAAATCAAA
Clone catalog no. AM043105
Original Clone ID: 414-4 LCVR7
Sequence Length: 336 bases
SEQ ID NO. 25
GATATTGTGATGACCCAGTCTCCACTCTCCCTGCCCGTCACCCCTGGAGA
GCCGGCCTCCATCTCCTGCAGGTCTAGTGAGAGCCTCCTGCATAGTACTG
GATACACGTATTTAGATTGGTTCGTGCAGAAGCCAGGGCAGTCTCCACGA
CTCCTGATCTATTTGGGTTATGATCGGGCCTCCGGGGTCCCTGACAGATT
CAGTGGCAGTGGAACAGGCACAGATTTCACGCTGAGAATCAGCAGAGTGG
AGCCTGAGGATGTTGGCATTTATTACTGCATGCAAGGTATACAAACTCCC
TACACTTTTGGCCAGGGGACCAAAGTGGATATCAAA
Clone catalog no. AM005415
Original Clone ID: 415-5 LCVR8
Sequence Length: 336 bases
SEQ ID NO. 26
CAGTCTGCCCTGACTCAGCCGCCCTCAGTGTCTGGGGCCCCAGGGCAGAG
GGTCACCATCTCCTGCACTGGGAGCAGCTCCAACATCGGGGCAGGTTATG
ATGTACACTGGTACCAGCAGCTTCCAGGAACAGCCCCCAAACTCCTCATC
TATGGTAACAGCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGGTTCG
AGGGTGTTCGGCGGAGGGACCGAGCTGACCGTCCTA
Clone catalog no. AM032414
Original Clone ID: 414-3-2 LCVR9
Sequence Length: 330 bases
SEQ ID NO. 27
CAGACTGTGGTGACTCAGCCTGCCTCCGTGTCTGGGTCTCCTGGACAGTC
GATCACCATCTCCTGCACTGGAACCAGCAGTGACGTTGGTGGTTATAACT
ATGTCTCCTGGTACCAACAGCACCCAGGCAAAGCCCCCAAACTCATGATT
TATGATGTCAGTAAGCGGCCCTCAGGGGTTTCTAATCGCTTCTCTGGCTC
CAAGTCTGGCAACACGGCCTCCCTGACCATCTCTGGGCTCCAGGCTGAGG
ACGAGGCTGATTATTACTGCAGCTCATATACAAGCAGCAGCCCCTGGGTG
TTCGGCGGAGGGACCGAGCTGACCGTCCTA
Clone catalog no. AM002414
Original Clone ID: 414-2 LCVR1
SEQ ID NO. 28
DIVMTQPPSVSGAPGQRVTISCTGSSSNIGAGYDVHWYQQLPGTAPKWYG
NSNRPSGVPDRFSGSKSGTSASLAITGLQAEDEADYYCQSYDSSLSGSVF
GGGTKVEIK
Clone catalog no. AM038105
Original Clone ID: 105-38 LCVR2
SEQ ID NO. 29
DVVLTQPHSVSESPGKTVTISCTGSSGSIASNYVQWYQQRPGSAPTTVIY
EDNQRPSGVPDRFSGSIDSSSNSASLTISGLKTEDEADYYCQSYDSSTDV
VFGGGTELTVL
Clone catalog no. AM009105
Original Clone ID: 105-9 LCVR3
SEQ ID NO. 30
EIVMTQSPATLSVSPGERATLSCRASQSVSSNLAWYQQKPGQGPRLLIYG
ASTRATGVPARFSGSGSGTDFTLTISSLQSEDFAIYYCQHYNNWFTFGQG
TKVEIK
Clone catalog no. AM006415
Original Clone ID: 415-6 LCVR4
SEQ ID NO. 31
QSVLTQPPSVSGAPGQRVTISCTGSSSNIGAGYDVHWYQQLPGTAPKWYG
NSNRPSGVPDRFSGSKSGTSASLAITGLQAEDEADYYCQSYDSSLSGVFG
RGTELTVL
Clone catalog no. AM001414
Original Clone ID: 414-1 LCVR5
SEQ ID NO. 32
QAGLTQPPSASGTPGQRVTISCSGSSSNIGSNTVNWYQQLPGTAPKWYSN
NQRPSGVPDRFSGSKSGTSASLAISGLQSEDEADYYCAAWDDSLNGVVFG
GGTELTVL
Clone catalog no. AM043105
Original Clone ID: 105-43 LCVR6
SEQ ID NO. 33
EIVMTQSPVTLSLSPGERATLSCRASQSVGTYLAWYQQKPGQAPRLLIYD
ASNRATRIPARFSGSGSGTDFTLTISSLEPEDFAVYYCQQGSNWPLTFGG
GTKVEIK
Clone catalog no. AM043105
Original Clone ID: 414-4 LCVR7
SEQ ID NO. 34
DIVMTQSPLSLPVTPGEPASISCRSSESLLHSTGYTYLDWFVQKPGQSPR
LLIYLGYDRASGVPDRFSGSGTGTDFTLRISRVEPEDVGIYYCMQGIQTP
YTFGQGTKVDIK
Clone catalog no. AM005415
Original Clone ID: 415-5 LCVR8
SEQ ID NO. 35
QSALTQPPSVSGAPGQRVTISCTGSSSNIGAGYDVHWYQQLPGTAPKWYG
NSNRPSGVPDRFSGSKSGTSASLAITGLQAEDEADYYCQSYDSSLSGSRV
FGGGTELTVL
Clone catalog no. AM032414
Original Clone ID: 414-3-2 LCVR9
SEQ ID NO. 36
QTVVTQPASVSGSPGQSITISCTGTSSDVGGYNYVSWYQQHPGKAPKLMI
YDVSKRPSGVSNRFSGSKSGNTASLTISGLQAEDEADYYCSSYTSSSPWV
FGGGTELTVL
Heavy Chain Constant Region
Sequence Length: 993 bases
SEQ ID NO. 37
GCTAGCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAAGTTGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGATGAGCTGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCTCCGGGTAAATGA
Heavy Chain Constant Region
SEQ ID NO. 38
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Light Chain Constant Region:
Sequence Length: 324 bases
SEQ ID NO. 39
CGAACGGTGGCTGCACCATCTGTCTTCATCTTCCCGCCATCTGATGAGCA
GTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGT
AACTCCCAGGAGAGTGTCACAGAGCAGGACAGCAAGGACAGCACCTACAG
CCTCAGCAGCACCCTGACGCTGAGCAAAGCAGACTACGAGAAACACAAAG
TCTACGCCTGCGAAGTCACCCATCAGGGCCTGAGCTCGCCCGTCACAAAG
AGCTTCAACAGGGGAGAGTGTTAG
Light Chain Constant Region:
SEQ ID NO. 40
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
COVID1 9 Spike Glycoprotein Receptor Binding
Domain
SEQ ID NO. 41
NITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPC
NGVEGFNCYFPLQSYGFQPTNGVGYQPYRVVVLSFELLHAPATV

What is claimed is:

1. A recombinant monoclonal antibody or antigen-binding fragment thereof that specifically binds to Severe Respiratory Syndrome Coronavirus-2 (SARS-CoV-2, COVID-19) spike protein, wherein the antibody or antigen-binding fragment comprises:
three heavy chain complementarity determining regions (CDRs) (HCDRI, HCDR2 and HCDR3) contained within any one of the heavy chain variable region (HCVR) sequences selected from the group consisting of SEQ ID NOs: -10-18; and
three light chain CDRs (LCDRI, LCDR2 and LCDR3) contained within any one of the light chain variable region (LCVR) sequences selected from the group consisting of SEQ ID NOs: -28-36,
wherein the antibody or antigen-binding fragment comprises a HCVR/LCVR amino acid sequence pair selected from the group consisting of SEQ ID NOs: 10/28, 11/29, 12/30, 13/31, 14/32, 15/33, 16/34, 17/35, 18/36.

2. The antibody or antigen-binding fragment thereof of claim 1, wherein the HCVR/LCVR amino acid sequence pairs are combined with heavy and light chain constant regions comprised of SEQ ID NO. 37 and 39, respectively.

3. The antibody or antigen-binding fragment thereof of claim 1, wherein the antibody has one or more of the following characteristics:
(a.) interacts with one or more amino acid residues in the receptor binding domain of the SARS-CoV-2 spike protein selected from amino acid residues 331-524 of SEQ ID NO 41;
(b.) blocks binding of SARS-CoV-2 spike protein to angiotensin converting enzyme 2 (ACE2) cellular receptor by more than 90%, as measured in a blocking ELISA assay;
(c.) neutralizes SARS-CoV-2 infectivity of human host cells by more than 90% and with IC50 less than 20 nM, as measured in a virus-like particle (VLP) neutralization assay;
(d.) neutralizes SARS-CoV-2 infectivity wherein the SARS-CoV-2 comprises an isolate of the virus derived from infected individuals;
(e.) prevents entry of SARS-CoV-2 into a host cell.

4. The antibody or antigen-binding fragment thereof of claim 1, comprising a HCVR having an amino acid sequence selected from the group consisting of SEQ ID NOs: 10-18.

5. The antibody or antigen-binding fragment thereof of claim 1, comprising a LCVR having an amino acid sequence selected from the group consisting of SEQ ID NOs: 28-36.

6. The antibody or antigen-binding fragment thereof of claim 1 which is a human antibody, a humanized antibody, or a chimeric antibody.

7. The antibody or antigen-binding fragment thereof of claim 1 which is a bi-specific antibody.

8. The antibody or antigen-binding fragment thereof of claim 7, wherein the antibody or antigen-binding fragment thereof is specific for a first epitope and a second, different, epitope of SARS-CoV-2 spike protein.

9. An isolated human antibody or antigen-binding fragment thereof that competes for binding to SARS-CoV-2 with the antibody or antigen-binding fragment of claim 1.

10. The antibody or antigen-binding fragment thereof of claim 9, wherein the antibody or antigen-binding fragment thereof interacts with the spike protein of patient-derived SARS-CoV-2 isolates.

11. The antibody or antigen-binding fragment thereof of claim 10, wherein the antibody or antigen-binding fragment thereof blocks the binding of SARS-CoV-2 to ACE2 on human cells.

12. The antibody or antigen-binding fragment thereof of claim 11, wherein the antibody or antigen-binding fragment thereof is produced as a recombinant antibody in mammalian expression host, preferably 293T cells or Chinese hamster ovary (CHO) cell lines.

13. An isolated recombinant human monoclonal antibody or antigen-binding fragment thereof that binds to the same epitope as the antibody or antigen-binding fragment of claim 1.

14. The antibody or antigen-binding fragment of claim 13, which is a human antibody, a humanized antibody, or a chimeric antibody.

15. The antibody or antigen-binding fragment of claim 13, which antibody recognizes an epitope comprising amino acid residues 331-524 of S1 Spike protein.

16. A recombinant monoclonal antibody or antigen-binding fragment thereof that specifically binds to Severe Respiratory Syndrome Coronavirus-2 (SARS-CoV-2, COVID-19) spike protein, wherein the antibody or antigen-binding fragment comprises:
(a) a HCDRI domain having an amino acid sequence selected from the group consisting of SEQ ID NOs: 10-18;
(b) a HCDR2 domain having an amino acid sequence selected from the group consisting of SEQ ID NOs: 10-18;
(c) a HCDR3 domain having an amino acid sequence selected from the group consisting of SEQ ID NOs: 10-18;
(d) a LCDRI domain having an amino acid sequence selected from the group consisting of SEQ ID NOs: 28-36;
(e) a LCDR2 domain having an amino acid sequence selected from the group consisting of SEQ ID NOs: 28-36;
(f) a LCDR3 domain having an amino acid sequence selected from the group consisting of SEQ ID NOs: 28-36

17. A pharmaceutical composition comprising the antibody or antigen-binding fragment thereof of claim 1 and a pharmaceutically acceptable carrier or diluent.

18. A pharmaceutical composition comprising:
(a) a first antibody or antigen-binding fragment thereof that binds to SARS-CoV-2 spike protein at a first epitope;
(b) a second antibody or antigen-binding fragment thereof that binds to SARS-CoV-2 spike protein at a second epitope; and
(c) a pharmaceutically acceptable carrier or diluent, wherein the first antibody or antigen-binding fragment thereof is an antibody or antigen-binding fragment comprising a HCVR/LCVR amino acid sequence pair selected from the group consisting of SEQ ID NOs: 10/28, 11/29, 12/30, 13/31, 14/32, 15/33, 16/34, 17/35, 18/36.

19. The pharmaceutical composition of claim 18, wherein at least the first antibody or antigen-binding fragment thereof or the second antibody or antigen-binding fragment thereof blocks SARS-CoV-2 binding to ACE2.

20. The pharmaceutical composition of claim 18, wherein the first and second epitopes are present in the receptor binding domain of COVID-19 spike protein and are distinct and non-overlapping.

21. The pharmaceutical composition of claim 20, wherein the first antibody or antigen-binding fragment thereof comprises:
three heavy chain complementarity determining regions (CDRs) (HCDRI, HCDR2 and HCDR3) contained within any one of the heavy chain variable region (HCVR) sequences selected from the group consisting of SEQ ID NOs: 10-18; and
three light chain CDRs (LCDRI, LCDR2 and LCDR3) contained within any one of the light chain variable region (LCVR) sequences selected from the group consisting of SEQ ID NOs: 28-36,
wherein the antibody or antigen-binding fragment comprises a HCVR/LCVR amino acid sequence pair selected from the group consisting of SEQ ID NOs: 10/28, 11/29, 12/30, 13/31, 14/32, 15/33, 16/34, 17/35, 18/36.

22. Kits and in vitro diagnostics utilizing one or more antibody or antigen-binding fragments thereof of claim 1 for the isolation and/or detection of SARS-CoV-2 in one or more sample types of human or nonhuman origin, including: blood, plasma, serum, saliva, tears, cerebrospinal fluid, lymph, urine, feces, exhaled breath condensate, perspiration, amniotic fluid, exosomes, cell and tissue lysates.

23. The kits and in vitro diagnostics of claim 22 for the isolation and/or detection of SARS-CoV-2 a nonhuman animal, e.g., a pangolin, a bat, a civet, or a camel.

24. A solid support bearing one or more antibody or antigen-binding fragments thereof of claim 1.

25. The solid support of claim 24, wherein the support comprises a microporous filter, e.g., a hollow microfiber.

26. A filter device comprising an elongated, hollow tube comprising and entrance and an exit and, within the tube, a plurality of elongated, microporous fibers having an interior lumen extending along the length thereof, wherein the microporous fibers comprise, immobilized thereto, an antibody or antigen binding fragment that binds to a SARS-CoV-2 spike protein, as described herein.

27. A method of detecting SARS-CoV-2 comprising performing an immunoassay on a biological sample from a subject; wherein the immunoassay uses an antibody or antigen-binding fragment of an antibody of any of claims in 1-15.

28. The method of claim 27, wherein the immunoassay selected from radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS).

29. The method of claim 28, wherein the subject is a human or a nonhuman mammal, e.g., pangolin, a bat, a civet or a camel.

30. The use of an antibody or antigen-binding fragment as described herein in the preparation of a medicament for the treatment of a subject infected with SARS-CoV-2.

31. The use of an antibody or antigen-binding fragment as described herein in the preparation of a diagnostic for the detection of SARS-CoV-2 in a sample.

32. A method of treating a subject infected with SARS-CoV-2 comprising administering to the subject an effective amount of a pharmaceutical composition comprising an antibody or antigen-binding fragment thereof of any of claims 1-15 and a pharmaceutically acceptable carrier or diluent.

33. A method of treating a subject infected with SARS-CoV-2 comprising performing plasmapheresis on the subject using plasma filter comprising a microporous fiber having immobilized thereto an antibody of any of claims 1-15 wherein the antibody or the antigen-binding fragment of the antibody removes SARS-CoV-2 from the blood of the subject.

34. A recombinant nucleic acid molecule encoding an antibody or antigen-binding fragment thereof of any of claims 1-15.

35. The recombinant nucleic acid molecule of claim 34, comprising nucleotide sequences encoding:
three heavy chain complementarity determining regions (CDRs) (HCDRI, HCDR2 and HCDR3) contained within any one of the heavy chain variable region (HCVR) sequences selected from the group consisting of SEQ ID NOs: 10-18; and
three light chain CDRs (LCDRI, LCDR2 and LCDR3) contained within any one of the light chain variable region (LCVR) sequences selected from the group consisting of SEQ ID NOs: 28-36,
wherein the antibody or antigen-binding fragment comprises a HCVR/LCVR amino acid sequence pair selected from the group consisting of SEQ ID NOs: 10/28, 11/29, 12/30, 13/31, 14/32, 15/33, 16/34, 17/35, 18/36.

36. The recombinant nucleic acid molecule of claim 34, comprising a nucleotide sequence selected from any of SEQ ID NOs: 1-9 and SEQ ID NOs: 19-27.

37. The recombinant nucleic acid molecule of claim 34, comprising a pair of nucleotide sequences selected from SEQ ID NOs: 1/19, 2/20, 3/21, 4/22, 5/23, 6/24, 7/25, and 8/26.

38. A recombinant expression construct comprising an expression control sequence operatively linked to a recombinant nucleic acid molecule of any one of claims 34 to 37.

39. A recombinant host cell comprising recombinant nucleic acid molecule of claim 38.

40. A method of making an antibody or antigen-binding fragment of an antibody comprising culturing a recombinant host cell comprising a recombinant expression construct comprising an expression control sequence operatively linked to a recombinant nucleic acid molecule encoding an antibody or antigen biding fragment of an antibody that binds to a SARS-CoV-2 spike protein, as provided herein, wherein the host cell produces the antibody or antigen-binding fragment of an antibody, and isolating the antibody or antigen-binding fragment of an antibody from the cell.


SEQUENCE LISTING (41 sequences)

SEQ ID NO: 1 - DNA, 378 bp, Homo sapiens
  Original Clone ID 414-2 HCVR1
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggggccctggggtgtagtagtaccagctgctatcctaacaactttgactactggggccagggaaccctggtcaccgtctcctca

SEQ ID NO: 2 - DNA, 357 bp, Homo sapiens
  Original Clone ID 105-38 HCVR2
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagtgcagtggctacggggggaatttgactactggggccagggaaccctggtcaccgtctcctca

SEQ ID NO: 3 - DNA, 363 bp, Homo sapiens
  Original Clone ID 105-9 HCVR3
gaagtgcagctggtggagtctgggggaggcttggtaaagccagggcggtccctgagactctcctgtacagcttctggattcacctttggtgattatgctatgagctggttccgccaggctccagggaaggggctggagtgggtaggtttcattagaagcaaagcatatggttggacaacacaatatgccgcgtctgtgaaaggcagattcaccatctcaagagatgattccaaaagcatcgcctatctgcaaatgaagagcctgaacagcgaggacacagccgtgtattactgtactagagatccatggggcactacgtactttgactactggggccagggaaccctggtcaccgtctcctca

SEQ ID NO: 4 - DNA, 369 bp, Homo sapiens
  Original Clone ID 415-6 HCVR4
caggtgcagctggtgcaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagatggatacagctatggttatccccacaactggttcgacccctggggccagggaaccctggtcaccgtctcctca

SEQ ID NO: 5 - DNA, 378 bp, Homo sapiens; identical to SEQ ID NO: 1
  Original Clone ID 414-1 HCVR5

SEQ ID NO: 6 - DNA, 345 bp, Homo sapiens
  Original Clone ID 105-43 HCVR6
gaagtgcagctggtggagtctgggggaggcttggtacagccgggggggtccctgagactctcctgtgcagcctcgggattcaccttcagtagttacgacatgcactgggtccgccaagctacaggaaaaggtctggaatggatctcagctattggtactactggtgacatatactatgcagactccgtgaagggccgattcaccatctccagagaaaatgccgagaactccttgtatcttcaaatgaacagcctgagagccggggacacggctgtgtattactgtgcaagatataggggtgcttttgatgtctggggccaagggacaatggtcaccgtctcttca

SEQ ID NO: 7 - DNA, 348 bp, Homo sapiens
  Original Clone ID 414-4 HCVR7
caggtgcagctggtgcaatctgagtctgagttgaagaagcctggggcctcagtgaagatttcctgcaagacttctggatacaccttcactaaatactttatgaattgggtgcgacaggcccctggacaagggcttgagtggctgggatggattaacaccgtcagtgggaacccaacctatgcccacggcttcacagggcagtttgtcttctccttggacgtctctatcaacacggcatatcttcagatcaacaacctaaagcctgcggacactggcgtctatttctgtgcgacagatattattccgagggactactggggccagggaaccctggtcaccgtctcctca

SEQ ID NO: 8 - DNA, 384 bp, Homo sapiens
  Original Clone ID 415-5 HCVR8
caggtgcagctggtggagtctggggctgaggtgaagaagcctgggtcctcggtgaaggtctcctgcaaggcttctggaggcaccttcaggagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagggccgtgtattactatgatagtagtgggtacccatacgccgaagactactttgactactggggccagggaaccctggtcaccgtctcctca

SEQ ID NO: 9 - DNA, 351 bp, Homo sapiens
  Original Clone ID 414-3-2 HCVR9
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcacttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagacggcgggggctggttcgacccctggggccagggaaccctggtcaccgtctcctca

SEQ ID NO: 10 - protein, 126 aa, Homo sapiens
  Original Clone ID 414-2 HCVR1
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGALGCSSTSCYPNNFDYWGQGTLVTVSS

SEQ ID NO: 11 - protein, 119 aa, Homo sapiens
  Original Clone ID 105-38 HCVR2
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARVQWLRGEFDYWGQGTLVTVSS

SEQ ID NO: 12 - protein, 121 aa, Homo sapiens
  Original Clone ID 105-9 HCVR3
EVQLVESGGGLVKPGRSLRLSCTASGFTFGDYAMSWFRQAPGKGLEWVGFIRSKAYGWTTQYAASVKGRFTISRDDSKSIAYLQMKSLNSEDTAVYYCTRDPWGTTYFDYWGQGTLVTVSS

SEQ ID NO: 13 - protein, 123 aa, Homo sapiens
  Original Clone ID 415-6 HCVR4
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSYGYPHNWFDPWGQGTLVTVSS

SEQ ID NO: 14 - protein, 126 aa, Homo sapiens; identical to SEQ ID NO: 10
  Original Clone ID 414-1 HCVR5

SEQ ID NO: 15 - protein, 115 aa, Homo sapiens
  Original Clone ID 105-43 HCVR6
EVQLVESGGGLVQPGGSLRLSCAASGFTFSSYDMHWVRQATGKGLEWISAIGTTGDIYYADSVKGRFTISRENAENSLYLQMNSLRAGDTAVYYCARYRGAFDVWGQGTMVTVSS

SEQ ID NO: 16 - protein, 116 aa, Homo sapiens
  Original Clone ID 414-4 HCVR7
QVQLVQSESELKKPGASVKISCKTSGYTFTKYFMNWVRQAPGQGLEWLGWINTVSGNPTYAHGFTGQFVFSLDVSINTAYLQINNLKPADTGVYFCATDIIPRDYWGQGTLVTVSS

SEQ ID NO: 17 - protein, 128 aa, Homo sapiens
  Original Clone ID 415-5 HCVR8
QVQLVESGAEVKKPGSSVKVSCKASGGTFRSYAISWVRQAPGQGLEWMGGIIPIFGTANYAQKFQGRVTITADESTSTAYMELSSLRSEDTAVYYCARAVYYYDSSGYPYAEDYFDYWGQGTLVTVSS

SEQ ID NO: 18 - protein, 117 aa, Homo sapiens
  Original Clone ID 414-3-2 HCVR9
QVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARDGGGWFDPWGQGTLVTVSS

SEQ ID NO: 19 - DNA, 333 bp, Homo sapiens
  Original Clone ID 414-2 LCVR1
gatattgtgatgactcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttcggtgttcggcggagggaccaaggtggaaatcaaa

SEQ ID NO: 20 - DNA, 333 bp, Homo sapiens
  Original Clone ID 105-38 LCVR2
gatgttgtgctgacacagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaccgatgtggtattcggcggagggaccgagctgaccgtccta

SEQ ID NO: 21 - DNA, 318 bp, Homo sapiens
  Original Clone ID 105-9 LCVR3
gaaatagtgatgacgcagtctccagccaccctgtctgtgtctccaggggaaagagccaccctctcctgcagggccagtcagagtgttagcagcaacttagcctggtaccagcagaaacctggccagggtcccaggctcctcatctatggtgcatccaccagggccactggtgtcccagccaggttcagtggcagtgggtctgggacagacttcactctcaccatcagcagcctgcagtctgaagattttgcaatttattactgtcagcactataataactggttcacttttggccaggggaccaaggtggagatcaaa

SEQ ID NO: 22 - DNA, 330 bp, Homo sapiens
  Original Clone ID 415-6 LCVR4
cagtctgtgctgactcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggggtgttcggcagagggaccgagctgaccgtccta

SEQ ID NO: 23 - DNA, 330 bp, Homo sapiens
  Original Clone ID 414-1 LCVR5
caggcagggctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccgagctgaccgtccta

SEQ ID NO: 24 - DNA, 321 bp, Homo sapiens
  Original Clone ID 105-43 LCVR6
gaaatagtgatgacgcagtctccagtcaccctgtctttgtctccaggggaaagagccaccctctcctgcagggccagtcagagtgttggcacctacttagcctggtaccaacagaaacctggccaggctcccaggctcctcatctatgatgcatccaacagggccactcgcatcccagccaggttcagtggcagtgggtctgggacagacttcactctcaccatcagcagcctagagcctgaagattttgcagtttattactgtcagcagggtagcaactggccgctcactttcggcggagggaccaaggtggaaatcaaa

SEQ ID NO: 25 - DNA, 336 bp, Homo sapiens
  Original Clone ID 414-4 LCVR7
gatattgtgatgacccagtctccactctccctgcccgtcacccctggagagccggcctccatctcctgcaggtctagtgagagcctcctgcatagtactggatacacgtatttagattggttcgtgcagaagccagggcagtctccacgactcctgatctatttgggttatgatcgggcctccggggtccctgacagattcagtggcagtggaacaggcacagatttcacgctgagaatcagcagagtggagcctgaggatgttggcatttattactgcatgcaaggtatacaaactccctacacttttggccaggggaccaaagtggatatcaaa

SEQ ID NO: 26 - DNA, 336 bp, Homo sapiens
  Original Clone ID 415-5 LCVR8
cagtctgccctgactcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttcgagggtgttcggcggagggaccgagctgaccgtccta

SEQ ID NO: 27 - DNA, 330 bp, Homo sapiens
  Original Clone ID 414-3-2 LCVR9
cagactgtggtgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcagcagcccctgggtgttcggcggagggaccgagctgaccgtccta

SEQ ID NO: 28 - protein, 111 aa, Homo sapiens
  Original Clone ID 414-2 LCVR1
DIVMTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKVEIK

SEQ ID NO: 29 - protein, 111 aa, Homo sapiens
  Original Clone ID 105-38 LCVR2
DVVLTQPHSVSESPGKTVTISCTGSSGSIASNYVQWYQQRPGSAPTTVIYEDNQRPSGVPDRFSGSIDSSSNSASLTISGLKTEDEADYYCQSYDSSTDVVFGGGTELTVL

SEQ ID NO: 30 - protein, 106 aa, Homo sapiens
  Original Clone ID 105-9 LCVR3
EIVMTQSPATLSVSPGERATLSCRASQSVSSNLAWYQQKPGQGPRLLIYGASTRATGVPARFSGSGSGTDFTLTISSLQSEDFAIYYCQHYNNWFTFGQGTKVEIK

SEQ ID NO: 31 - protein, 110 aa, Homo sapiens
  Original Clone ID 415-6 LCVR4
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGVFGRGTELTVL

SEQ ID NO: 32 - protein, 110 aa, Homo sapiens
  Original Clone ID 414-1 LCVR5
QAGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTELTVL

SEQ ID NO: 33 - protein, 107 aa, Homo sapiens
  Original Clone ID 105-43 LCVR6
EIVMTQSPVTLSLSPGERATLSCRASQSVGTYLAWYQQKPGQAPRLLIYDASNRATRIPARFSGSGSGTDFTLTISSLEPEDFAVYYCQQGSNWPLTFGGGTKVEIK

SEQ ID NO: 34 - protein, 112 aa, Homo sapiens
  Original Clone ID 414-4 LCVR7
DIVMTQSPLSLPVTPGEPASISCRSSESLLHSTGYTYLDWFVQKPGQSPRLLIYLGYDRASGVPDRFSGSGTGTDFTLRISRVEPEDVGIYYCMQGIQTPYTFGQGTKVDIK

SEQ ID NO: 35 - protein, 112 aa, Homo sapiens
  Original Clone ID 415-5 LCVR8
QSALTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSRVFGGGTELTVL

SEQ ID NO: 36 - protein, 110 aa, Homo sapiens
  Original Clone ID 414-3-2 LCVR9
QTVVTQPASVSGSPGQSITISCTGTSSDVGGYNYVSWYQQHPGKAPKLMIYDVSKRPSGVSNRFSGSKSGNTASLTISGLQAEDEADYYCSSYTSSSPWVFGGGTELTVL

SEQ ID NO: 37 - DNA, 993 bp, Homo sapiens
  Heavy Chain Constant Region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 330 aa, Homo sapiens [sequence omitted]

SEQ ID NO: 39 - DNA, 324 bp, Homo sapiens
  Light Chain Constant Region
cgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 107 aa, Homo sapiens [sequence omitted]

SEQ ID NO: 41 - protein, 194 aa, Severe acute respiratory syndrome coronavirus 2
  COVID19 Spike Glycoprotein Receptor Binding Domain
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV